(19) United States
(12) Patent Application Publication
(10) Pub. No.: US 2018/0280053 A1
(43) Pub. Date: Oct. 4, 2018

(54) Ultrasound Probe with Integrated Needle Advancer

(71) Applicant: Avent, Inc., Alpharetta, GA (US)

(72) Inventors: Justin J. Coker, Laguna Niguel, CA (US); Kenneth C. Hsu, Tustin, CA (US)

(21) Appl. No.: 15/765,826

(22) PCT Filed: Sep. 15, 2016
(86) PCT No.: PCT/US2016/051873

§ 371 Date: Apr. 4, 2018

Related U.S. Application Data
(60) Provisional application No. 62/247,891, filed on Oct. 29, 2015.

(51) Int. Cl. A61B 17/34; A61B 5/06; A61B 8/14; A61B 8/00; A61B 8/08

(52) U.S. Cl. CPC A61B 17/3403; A61B 5/065; A61B 8/14; A61B 8/4455; A61B 2090/062; A61B 8/467; A61B 8/5207; A61B 2017/3413; A61B 8/461

(57) ABSTRACT

The present disclosure is directed to an ultrasound imaging system with an integrated needle advancer. More specifically, the ultrasound imaging system includes an ultrasound probe having a transducer housing, a transducer transmitter, a needle assembly, and a controller. The transducer housing has a body that defines an internal cavity. The internal cavity includes a passageway extending from a proximal end to a distal end of the body. The needle assembly is configured within the passageway of the internal cavity. The transducer transmitter is configured within the distal end of the body. Further, the transducer transmitter is configured to emit and receive ultrasound beams. Thus, the controller is configured to generate an image from the ultrasound beams.

### RELATED APPLICATIONS

This application claims the benefit of U.S. Provisional Applications No. 62/247,891, filed Oct. 29, 2015, which is incorporated by reference herein in its entirety.

### FIELD OF THE INVENTION

The present invention relates in general to ultrasound imaging systems, and more particularly to a medical ultrasound probe with an integrated needle advancer, particularly useful for nerve block applications.

### BACKGROUND OF THE INVENTION

In conventional two-dimensional (2D) ultrasound imaging, a focused beam of ultrasound energy is transmitted into body tissues to be examined and the returned echoes are detected and plotted to form an image. Some modern ultrasound systems have three-dimensional (3D) capabilities that scan a pulsed ultrasound beam in two side-wards directions relative to a beam axis. Time of flight conversion gives the image resolution along the beam direction (range), while image resolution transverse to the beam direction is obtained by the side-wards scanning of the focused beam. With such 3D imaging, a user can collect volume ultrasound data from an object and visualize any cross-section of the object through computer processing. This enables selection of the best two-dimensional (2D) image planes for a diagnosis.

Thus, 2D or 3D ultrasound imaging is useful for numerous medical applications where visual aid is advantageous, e.g. nerve block applications. However, during some peripheral nerve block applications, it can be very difficult to keep both the nerve bundle and the needle in the plane with the ultrasound field. Thus, it can be very easy for the needle to be out of the ultrasound plane such that the ultrasound imaging cannot capture an image of the needle. When this happens, the needle cannot be seen, thereby creating a potentially dangerous situation for the patient if the needle is misplaced. In addition, the procedure can become very difficult for the anesthesiologist due to the requirement of having to hold, maintain, and operate multiple devices during the procedure.

Thus, the art is continuously seeking new and improved 2D or 3D ultrasound probes. More specifically, an ultrasound probe with 2D or 3D capabilities that has an integrated needle advancer that enhances the effectiveness of nerve block procedures would be advantageous.

### BRIEF SUMMARY OF THE INVENTION

Aspects and advantages of the invention will be set forth in part in the following description, or may be obvious from the description, or may be learned through practice of the invention.

In one aspect, the present disclosure is directed to an ultrasound imaging system. The ultrasound imaging system includes an ultrasound probe having 2D and/or 3D capabilities. More specifically, the ultrasound probe has a transducer housing, a transducer transmitter, a needle assembly, and a controller. The transducer housing has a body that defines an internal cavity. The internal cavity includes a passageway extending from a proximal end to a distal end of the body. Thus, the needle assembly is configured within the passageway of the internal cavity. In addition, the transducer transmitter is configured within the distal end of the body so as to emit and receive ultrasound beams. As such, the controller is configured to generate an image from the ultrasound beams.

In one embodiment, the image may be a two-dimensional (2D) image generated by a 2D probe. Alternatively, the image may be a three-dimensional (3D) image generated by a 3D probe.

In another embodiment, the needle assembly includes a needle extending from a proximal end to a distal end with a hub configured at the proximal end of the needle. Thus, in particular embodiments, the needle fits within the passageway of the internal cavity via an interference fit.

In another embodiment, the needle may be centrally located within the transducer housing. Thus, in such embodiments, the needle is configured to align with the ultrasound plane of the ultrasound probe during operation such that the insertion depth of the needle can be automatically and/or manually controlled by a user. For example, in certain embodiments, the controller of the imaging system may be configured to automatically control movement of the needle within the passageway. More specifically, the transducer housing may include one or more sensors configured therein so as to determine an insertion depth of the needle within a patient. Thus, the sensor(s) are configured to send signals to the controller, the signals being indicative of the insertion depth of the needle. Accordingly, the controller is configured to automatically control the insertion depth of the needle based on the sensor signals.

In an alternative embodiment, the ultrasound probe may include manual control features. For example, in one embodiment, the manual control features may include one or more control buttons configured on the transducer housing. Further, the control buttons are communicatively coupled with the controller. As such, engagement of the control buttons (e.g. by pressing or lifting the buttons) is configured to control an insertion depth of the needle.

In another embodiment, the ultrasound imaging system may include a user interface configured to display the image, e.g. either a 2D or 3D image. More specifically, the user interface is configured to allow a user to manipulate the image according to one or more user preferences.

In further embodiments, the distal end of the body of the transducer housing may have a lens having a linear configuration. In such an embodiment, the transducer transmitter is configured adjacent to the lens. In additional embodiments, the distal end of the body of the transducer housing is wider than the proximal end.

In another aspect, the present disclosure is directed to an ultrasound probe for imaging. The ultrasound probe includes a transducer housing, a needle assembly, and a transducer transmitter. The transducer housing has a body that defines an internal cavity. The internal cavity has a passageway extending from a proximal end to a distal end of the body. Thus, the needle assembly is configured within the passageway of the internal cavity. Further, the transducer transmitter is configured within the distal end of the body and is configured to emit and receive ultrasound beams so to generate an image. It should be understood that the ultrasound probe may be further configured with any of the additional features as described herein.

In yet another aspect, the present disclosure is directed to a method of generating an ultrasound image during a nerve block procedure. The method includes aligning an ultrasound probe at a target site of a patient. As mentioned, the ultrasound probe may include a transducer housing having a passageway extending from a proximal end to a distal end thereof and a transducer transmitter configured within the housing. As such, the method also includes inserting a needle assembly through the passageway of the transducer housing and into the patient towards the target site. In addition, the method includes emitting and receiving, via the transducer transmitter, ultrasound beams from the target site while the needle assembly is inserted in the patient. Thus, the method also includes generating an image based on the ultrasound beams.

In one embodiment, as mentioned, the needle assembly may include a needle extending from a proximal end to a distal end with a hub configured at the proximal end of the needle. As such, the step of inserting the needle assembly through the passageway of the transducer housing and into the patient towards the target site may include inserting the needle of the needle assembly through a central location of the transducer housing, e.g. such that the needle aligns with an ultrasound plane during operation.

In another embodiment, the method may include automatically controlling, via the controller, movement of the needle within the passageway. For example, in such an embodiment, the method may include determining, via one or more sensors configured with the transducer housing, an insertion depth of the needle within the patient. More specifically, in certain embodiments, the method may include sending, via the one or more sensors, signals to the controller that are indicative of the insertion depth of the needle and automatically controlling the insertion depth of the needle based on the signals.

Alternatively, the method may include manually controlling the needle within the passageway. More specifically, in certain embodiments, the step of manually controlling the needle within the passageway may include engaging (e.g. by pressing or lifting) one or more control buttons configured on the transducer housing. As mentioned, the control buttons are communicatively coupled with the controller such that engagement of the control button(s) controls an insertion depth of the needle.

In another embodiment, the method may include displaying, via a user interface, the image to a user, e.g. either a 2D or 3D image. More specifically, in certain embodiments, the method may include allowing, via the user interface, a user to manipulate the image according to one or more user preferences.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 illustrates a schematic diagram of one embodiment of an ultrasound imaging system according to the present disclosure;

FIG. 2 illustrates a block diagram of one embodiment of suitable components that may be included in a controller of an ultrasound imaging system according to the present disclosure;

FIG. 3 illustrates a front, perspective view of one embodiment of an ultrasound probe of an ultrasound imaging system according to the present disclosure;

FIG. 4 illustrates a detailed view of the ultrasound probe of FIG. 3;

FIG. 5 illustrates an internal, detailed view of one embodiment of an ultrasound probe of an ultrasound imaging system according to the present disclosure, particularly illustrating the probe located at a target site of a patient;

FIG. 6 illustrates an internal, detailed view of another embodiment of an ultrasound of an ultrasound imaging system according to the present disclosure, particularly illustrating the probe located at a target site of a patient and an integrated needle assembly configured therethrough;

FIG. 7 illustrates a front, perspective view of one embodiment of an ultrasound probe of an ultrasound imaging system according to the present disclosure, particularly illustrating an integrated needle assembly configured within a passageway of the transducer housing of the ultrasound probe; and

FIG. 8 illustrates a flow diagram of one embodiment of a method of generating an ultrasound image according to the present disclosure.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

Generally, the present disclosure is directed to an improved ultrasound probe with an integrated needle advancer. More specifically, the ultrasound probe has a transducer housing with a transducer transmitter configured therein, a needle assembly integrated within the transducer housing, and a controller. The transducer housing has a body that defines an internal cavity with a passageway extending from a proximal end to a distal end thereof. Thus, the needle assembly is configured within the passageway of the internal cavity. Further, the transducer transmitter is configured within the distal end of the body so as to emit and receive ultrasound beams. As such, the controller is configured to generate an image from the ultrasound beams. In addition, the controller is configured to control an insertion depth of the needle assembly.

Such a system can be particularly advantageous for various medical procedures, including for example, nerve block applications. More specifically, the ultrasound probe of the present disclosure can be placed at a target site of a patient (e.g. on an outer surface of the patient's skin where a nerve block procedure is to be performed at a nerve or nerve bundle therebeneath) and can remain in the same location as the needle assembly is advanced towards the nerve bundle and the transducer transmitter generates the ultrasound beams. As such, if desired, a user can manipulate the ultrasound probe and the needle with one hand. Further, the integrated needle remains in the ultrasound plane for easy viewing of a 2D or 3D image.

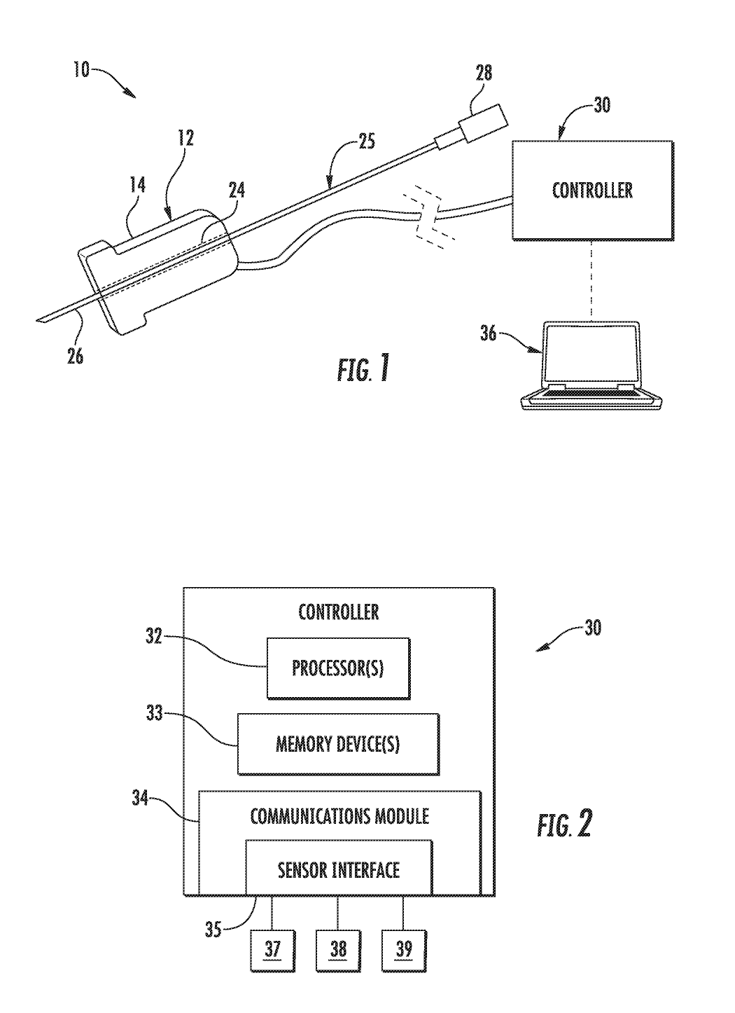

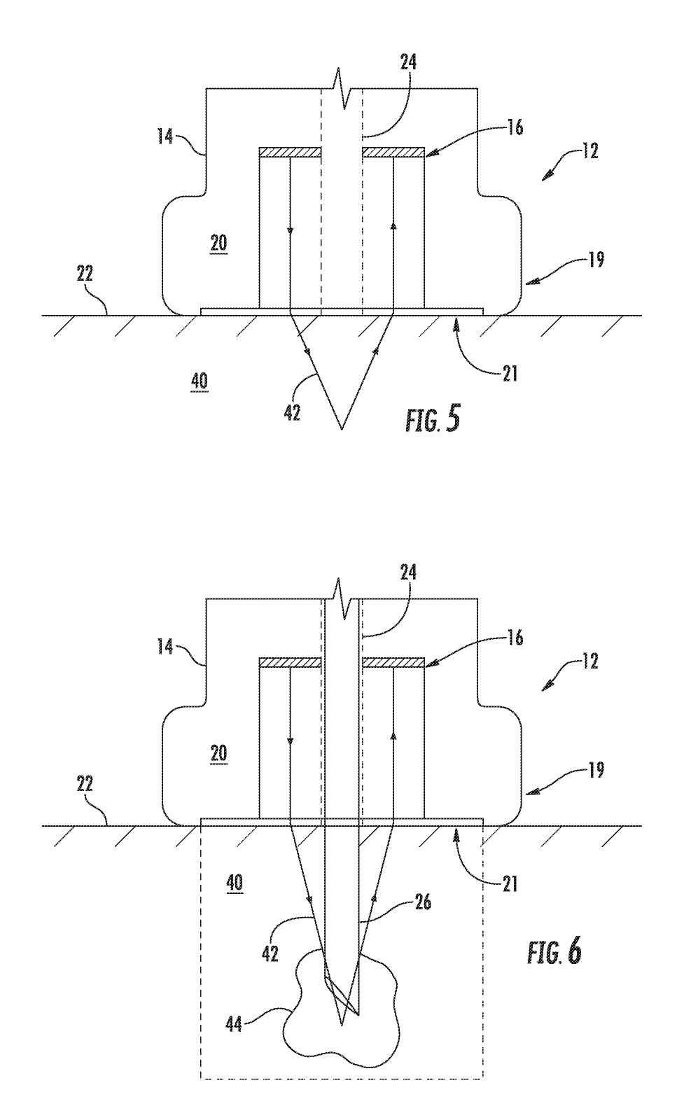

Referring now to the drawings, FIG. 1 illustrates a schematic diagram of one embodiment of an ultrasound imaging system 10 according to the present disclosure. As shown, the ultrasound imaging system 10 includes an ultrasound probe 12. More specifically, as shown in FIGS. 3-7, the ultrasound probe 12 has a transducer housing 14, a transducer transmitter 16, and a needle assembly 25 integrated within the housing 14. Further, as shown in FIGS. 3, 4 and 7, the housing 14 generally has a body 15 extending from a proximal end 17 to a distal end 19 along a longitudinal axis 18 thereof. In certain embodiments, as shown generally in the figures, the distal end 19 of the body 15 of the housing 14 may be wider than the proximal end 17 of the body 15, e.g. such that the proximal end 17 of the body 15 can be easily gripped by a user. Alternatively, the distal end 19 of the body 15 of the housing 14 may be narrower than the proximal end 17 of the body 15. In still another embodiment, the proximal and distal ends 17, 19 of the body 15 of the housing 14 may have substantially the same width along the longitudinal axis 18.

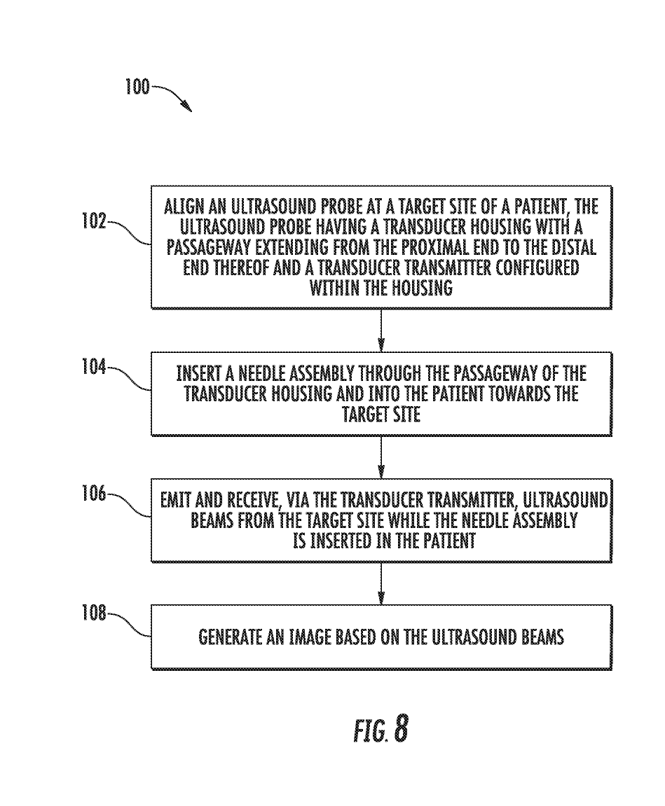

In addition, as shown, the body 15 defines an internal cavity 20 having a passageway 24 extending from the proximal end 17 to the distal end 19 of the body 15. In addition, as shown generally in the figures, the needle assembly 25 is configured within the passageway 24 of the internal cavity 20. More specifically, as shown, the needle assembly 25 includes a needle 26 extending from a proximal end to a distal end with a hub 28 configured at the proximal end of the needle 26. Further, in certain embodiments, the needle 26 is configured to fit within the passageway 24 of the internal cavity 20, e.g. via an interference fit. Alternatively, as shown in FIG. 7, the diameter of the needle 26 may be substantially less than the diameter of the passageway 24.

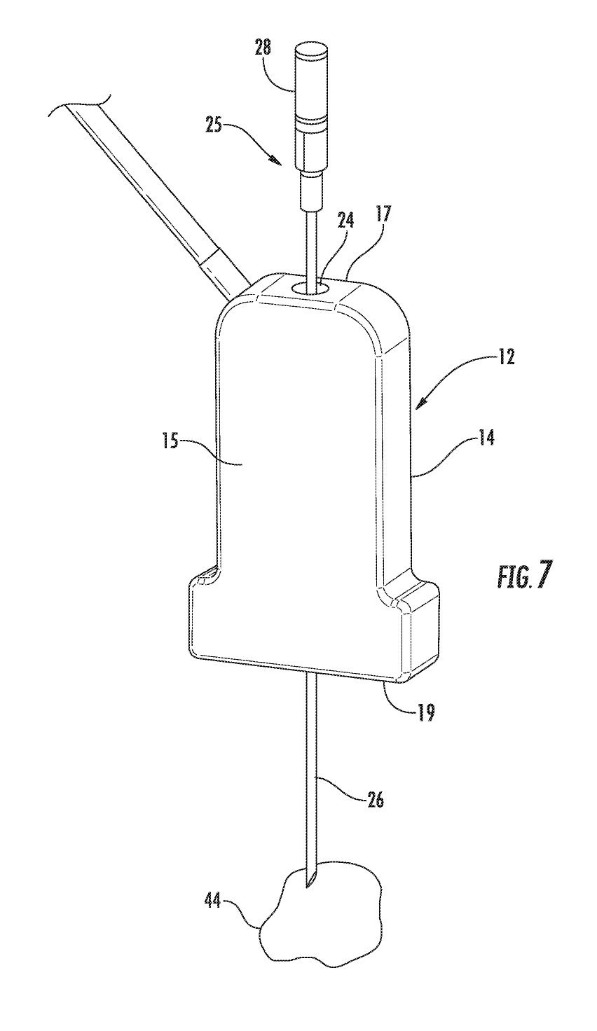

In further embodiments, as shown in FIGS. 5 and 6, the distal end 19 of the body 15 of the transducer housing 14 may also include a lens 21 having any suitable configuration. Thus, the lens 21 is configured to allow passage of the ultrasonic beams 42 therethrough. For example, as shown, the lens 21 may have a linear configuration. In further embodiments, the lens 21 may have a convex configuration. Thus, as shown, the transducer transmitter 16 may be configured adjacent to the lens 21.

As is generally understood, the transducer transmitter 16 is configured to emit and/or receive ultrasound beams. Thus, the transducer transmitter 16 may have any suitable configuration now known or later developed in the art. More specifically, during operation, the probe 12 can be placed at a target site 22 of the patient and while maintaining the probe 12 in its initial position, the transducer transmitter 16 is configured to continuously emit and receive ultrasound beams 42 in an ultrasound plane 40.

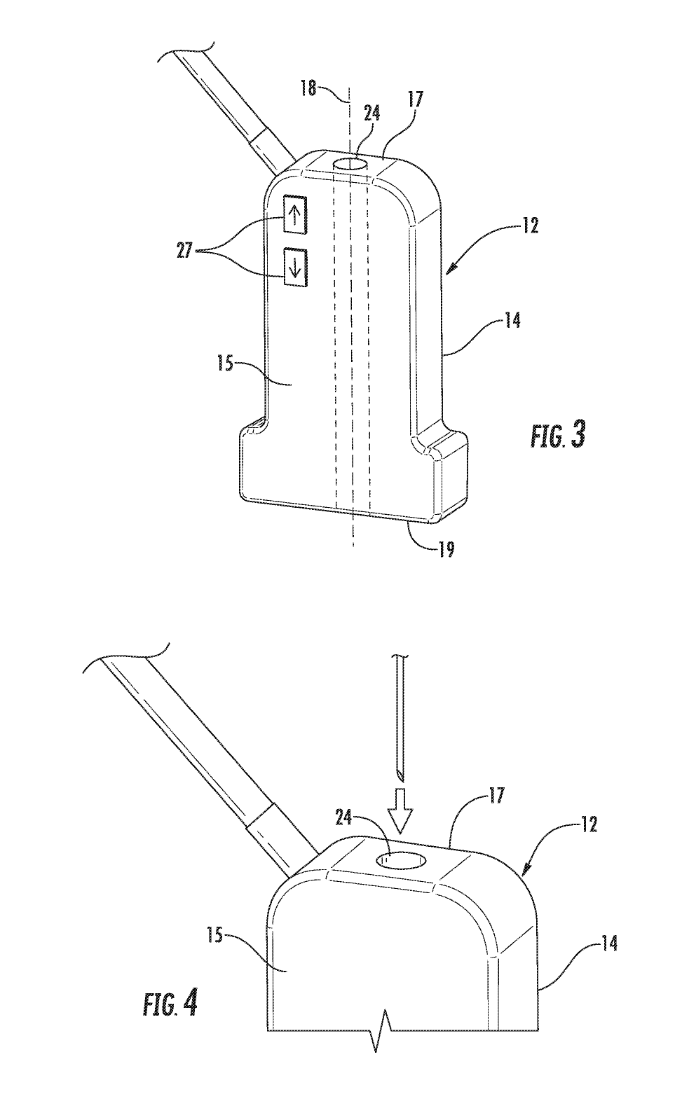

In further embodiments, as shown generally in the figures, the needle 26 may be centrally located within the transducer housing 14. Thus, as shown in FIGS. 5 and 6, the needle 26, when inserted at a target site 22, is aligned with the ultrasound plane 40 of the ultrasound probe 12 such that the insertion depth of the needle 26 can be automatically or manually controlled by a user. As such, the probe 12 can be particularly advantageous for nerve block applications. More specifically, as shown in FIGS. 1 and 2, the ultrasound imaging system 10 may include a controller 30 (FIG. 2) configured to automatically control movement of the needle 26 within the passageway 24. For example, in certain embodiments, one or more sensors 37, 38, 39 (FIG. 2) may be configured with the transducer housing 14 so as to determine a suitable insertion depth of the needle 26 within a patient at the target site 22. Thus, the sensor(s) 37, 38, 39 are configured to send signals to the controller 30 that are indicative of the insertion depth of the needle 26. Accordingly, the controller 30 is configured to automatically control the insertion depth of the needle 26 based on the sensor signals. In addition, the controller 30 is configured to receive and organize the ultrasound beams 42 generated by the transducer transmitter 16 in real-time and generate an ultrasound image based on the beams 42.

Referring specifically to FIG. 2, there is illustrated a block diagram of one embodiment of suitable components that may be included within the controller 30 in accordance with aspects of the present subject matter. As shown, the controller 30 may include one or more processor(s) 32 and associated memory device(s) 33 configured to perform a variety of computer-implemented functions (e.g., performing the methods, steps, and the like and storing relevant data as disclosed herein). Additionally, the controller 30 may also include a communications module 34 to facilitate communications between the controller 30 and the various components of the system 10. Further, the communications module 34 may include a sensor interface 35 (e.g., one or more analog-to-digital converters) to permit signals transmitted from the probe 12 to be converted into signals that can be understood and processed by the processors 32. In addition, as shown, the ultrasound imaging system 10 may also include a user interface 36 (FIG. 1) configured to display the image, e.g. either a 2D or 3D image. More specifically, in certain embodiments, the user interface 36 may be configured to allow a user to manipulate the image according to one or more user preferences.

In an alternative embodiment, the ultrasound probe 12 may include manual control features. For example, in one embodiment, the manual control features may include one or more control buttons 27 configured on the transducer housing 14. Further, the control buttons 27 may be communicatively coupled with the controller 30. As such, engagement of the control buttons 27 is configured to control an insertion depth of the needle 26, e.g. by moving the needle 26 up and down within the passageway 24. In still additional embodiments, the ultrasound probe 12 may be controlled using a combination of manual and/or automatic features.

Referring now to FIG. 8, a flow diagram of one embodiment of a method 100 of generating an ultrasound image is illustrated. As shown at 102, the method 100 includes aligning an ultrasound probe 12 at a target site 22 of a patient, e.g. adjacent to an insertion point for a nerve block procedure. Further, as mentioned, the ultrasound probe 12 may be a 2D or a 3D probe. More specifically, as mentioned, the ultrasound probe 12 may include a transducer housing 14 having a passageway 24 extending from the proximal end 17 to the distal end 19 thereof and a transducer transmitter 16 configured within the housing 14. Thus, as shown at 104, the method 100 includes inserting a needle assembly 25 through the passageway 24 of the transducer housing 14 and into the patient at the target site 22, e.g. towards a nerve or nerve bundle 44 (FIGS. 6-7). As shown at 106, the method 100 includes emitting and receiving, via the transducer transmitter 16, ultrasound beams 42 from the target site 22 while the needle assembly 25 is inserted in the patient. As shown at 108, the method 100 includes generating an image based on the ultrasound beams 42.

As mentioned, in one embodiment, the needle assembly 25 may include a needle 26 extending from a proximal end to a distal end with a hub 28 configured at the proximal end of the needle 26. As such, the step of inserting the needle assembly 25 through the passageway 24 of the transducer housing 14 and into the patient towards the target site 22 may include inserting the needle 26 of the needle assembly 25 through a central location of the transducer housing 14.

In another embodiment, the method 100 may include automatically controlling, via the controller 30, movement of the needle 26 within the passageway 24. For example, in such an embodiment, the method 100 may include determining, via one or more sensors 37, 38, 39 configured with the transducer housing 14, an insertion depth of the needle 26 within the patient. More specifically, in certain embodiments, the method 100 may include sending, via the one or more sensors 37, 38, 39, signals to the controller 30 that are indicative of the insertion depth of the needle 26 and automatically controlling the insertion depth of the needle 26 based on the signals, e.g. by moving the needle 26 up or down.

Alternatively, the method 100 may include manually controlling the needle 26 within the passageway 24. More specifically, in certain embodiments, the step of manually controlling the needle 26 within the passageway 24 may include engaging (e.g. by pressing or lifting) one or more control buttons 27 configured on the transducer housing 14. As mentioned, the control buttons 27 are communicatively coupled with the controller 30 such that engagement of the control button(s) 27 controls an insertion depth of the needle 26.

In another embodiment, the method 100 may include displaying, via a user interface 36, the image to a user, e.g. either a 2D or 3D image. More specifically, in certain embodiments, the method 100 may include allowing, via the user interface 36, a user to manipulate the image according to one or more user preferences.

While various patents have been incorporated herein by reference, to the extent there is any inconsistency between incorporated material and that of the written specification, the written specification shall control. In addition, while the disclosure has been described in detail with respect to specific embodiments thereof, it will be apparent to those skilled in the art that various alterations, modifications and other changes may be made to the disclosure without departing from the spirit and scope of the present disclosure. It is therefore intended that the claims cover all such modifications, alterations and other changes encompassed by the appended claims.

1. An ultrasound imaging system, comprising:
an ultrasound probe comprising:
a transducer housing comprising a body that defines an internal cavity, the internal cavity comprising a passageway extending from a proximal end to a distal end of the body;
a needle assembly configured within the passageway of the internal cavity;
a transducer transmitter configured within the distal end of the body, the transducer transmitter configured to emit and receive ultrasound beams; and
a controller configured to generate an image from the ultrasound beams.

2. The ultrasound imaging system of claim 1, wherein the needle assembly comprises a needle extending from a proximal end to a distal end and a hub configured at the proximal end of the needle.

3. The ultrasound imaging system of claim 2, wherein the needle is centrally located within the transducer housing.

4. The ultrasound imaging system of claim 2, wherein the needle fits within the passageway of the internal cavity via an interference fit.

5. The ultrasound imaging system of claim 1, wherein the controller is configured to automatically control movement of the needle within the passageway.

6. The ultrasound imaging system of claim 1, wherein the transducer housing further comprises one or more sensors configured to determine an insertion depth of the needle within a patient, wherein the one or more sensors are configured to send signals to the controller that are indicative of the insertion depth of the needle, wherein the controller is configured to automatically control the insertion depth of the needle based on the signals.

7. The ultrasound imaging system of claim 1, wherein the ultrasound probe comprises manual control features, wherein the manual control features comprise one or more control buttons configured on the transducer housing, the one or more control buttons communicatively coupled with the controller, wherein engagement of the one or more control buttons controls an insertion depth of the needle.

8. The ultrasound imaging system of claim 1, wherein the image comprises a two-dimensional (2D) image or a three-dimensional (3D) image.

9. The ultrasound imaging system of claim 1, further comprising a user interface configured to display the image, wherein the user interface is configured to allow a user to manipulate the image according to one or more user preferences.

10. An ultrasound probe for imaging, comprising:
a transducer housing comprising a body that defines an internal cavity, the internal cavity comprising a passageway extending from a proximal end to a distal end of the body;
a needle assembly configured within the passageway of the internal cavity; and
a transducer transmitter configured within the distal end of the body, the transducer transmitter configured to emit and receive ultrasound beams so to generate an image.

11. The ultrasound probe of claim 10, wherein the needle assembly comprises a needle extending from a proximal end to a distal end and a hub configured at the proximal end of the needle.

12. The ultrasound probe of claim 11, wherein the needle is centrally located within the transducer housing.

13. The ultrasound probe of claim 10, wherein the needle fits within the passageway of the internal cavity via an interference fit.

14. The ultrasound probe of claim 10, wherein the transducer housing further comprises one or more sensors configured to determine an insertion depth of the needle within a patient.

15. The ultrasound probe of claim 14, wherein the one or more sensors are configured to send signals to a controller, the signals being indicative of the insertion depth of the needle, wherein the controller is configured to automatically control the insertion depth of the needle based on the signals.

16. The ultrasound probe of claim 10, further comprising manual control features, wherein the manual control features comprise one or more control buttons configured on the transducer housing, the one or more control buttons communicatively coupled with a controller, wherein engagement of the one or more control buttons controls an insertion depth of the needle.

17. A method of generating an ultrasound image during a nerve block procedure, the method comprising:
aligning an ultrasound probe at a target site of a patient, the ultrasound probe having a transducer housing comprising a passageway extending from a proximal end to a distal end thereof and a transducer transmitter;
inserting a needle assembly through the passageway of the transducer housing and into the patient towards the target site, the needle assembly comprising a needle extending from a proximal end to a distal end;
emitting and receiving, via the transducer transmitter, ultrasound beams from the target site while the needle assembly is inserted in the patient; and
generating an image based on the ultrasound beams.

18. The method of claim 17, wherein inserting the needle assembly through the passageway of the transducer housing and into the patient towards the target site further comprises inserting the needle of the needle assembly through a central location of the transducer housing.

19. The method of claim 17, further comprising automatically controlling, via the controller, movement of the needle within the passageway.

20. The method of claim 17, further comprising:
determining, via one or more sensors configured with the transducer housing, an insertion depth of the needle within the patient;
sending, via the one or more sensors, signals to the controller that are indicative of the insertion depth of the needle; and
controlling the insertion depth of the needle based on the signals.
